(19) United States
(12) Patent Application Publication
(10) Pub. No.: US 2024/0273784 A1
(43) Pub. Date: Aug. 15, 2024

(54) X-Ray Imaging System and Image Processing Method

(71) Applicant: Shimadzu Corporation, Kyoto-shi (JP)

(72) Inventor: Tetsuya KOBAYASHI, Kyoto-shi (JP)

(21) Appl. No.: 18/290,690

(22) PCT Filed: May 19, 2022
(86) PCT No.: PCT/JP2022/020774

§ 371 Date: Jan. 19, 2024

(30) Foreign Application Priority Data
Jul. 20, 2021 (JP) 2021-119743

(51) Int. Cl. G06T 11/00; A61B 6/00; A61B 6/02; A61B 6/50

(52) U.S. Cl. CPC G06T 11/006; A61B 6/025; A61B 6/505; A61B 6/5252; G06T 11/008; G06T 2210/41; G06T 2211/441

(57) ABSTRACT

An X-ray imaging system (100) according to this invention includes an image processor (9) configured to generate a bone-suppressed tomographic image (20) representing a cross-section of a subject (101) in which a bone structure of a target part is suppressed based on a plurality of X-ray images (10). The image processor (9) includes a bone suppression processing unit (92) configured to suppress the bone structure of the target part, a reconstruction processing unit (93) configured to perform reconstruction for generating the tomographic image, and an adjustment processing unit (94) configured to adjust a suppression degree of the bone structure in the bone-suppressed tomographic image (20) to be generated.

### TECHNICAL FIELD

The present invention relates to an X-ray imaging system and an image processing method.

### BACKGROUND ART

Tomographic image generation systems for capturing tomosynthesis images are known in the art. Such a system is disclosed in Japanese Patent Laid-Open Publication No. JP 2016-22095, for example.

The tomographic imaging system disclosed in the above Japanese Patent Laid-Open Publication No. JP 2016-22095 includes a radiographic device and a console. The radiographic device includes a radiation source for emitting radiation, and a radiation detector for detecting the radiation. The radiographic device is configured to take a plurality of tomosynthesis imaging shots while the radiation source and the radiation detector are synchronously moved to acquire a projected image in each shot. The console then reconstructs the projection images acquired by the radiographic device to generate a reconstructed image of an object to be captured.

### PRIOR ART

### Patent Document

Patent Document 1: Japanese Patent Laid-Open Publication No. JP 2016-22095

### SUMMARY OF THE INVENTION

### Problems to be Solved by the Invention

Although not stated in the above Japanese Patent Laid-Open Publication No. JP 2016-22095, an angle range of radiation emitted to capture an image in tomosynthesis imaging is limited as compared with CT imaging, and as a result a false image (artifact) of a bone (bone structure) may occur in such a reconstructed image (tomographic image) generated. For example, in a case in which a tomographic image of lungs in a cross-section is generated, a false image of ribs may appear in the cross-section of the lungs. In this case, the false image of ribs deteriorates visibility of a lesion part in the lungs. Consequently, if such a bone artifact appears in a part in which no bone actually exists in the generated tomographic image, the bone artifact deteriorates visibility of a target part of in a body of an object to be captured (subject).

The present invention is intended to solve the above problem, and one object of the present invention is to provide an X-ray imaging system and an image processing method capable of preventing deterioration of visibility of a target part caused by an artifact of a bone in generation of a tomographic image showing a cross-section of a subject.

### Means for Solving the Problems

In order to attain the aforementioned object, an X-ray imaging system according to a first aspect of the present invention includes an X-ray irradiator configured to irradiate a target part of a subject with X-rays; an X-ray detector configured to detect the X-rays with which the subject is irradiated by the X-ray irradiator; a moving mechanism configured to move at least one of the X-ray irradiator and the X-ray detector; an imaging controller configured to perform X-ray imaging of the target part of the subject while the moving mechanism is the at least one of the X-ray irradiator and the X-ray detector; and an image processor configured to generate, based on a plurality of X-ray images generated by the X-ray imaging, a bone-suppressed tomographic image representing a cross-section of the subject in which a bone structure of the target part is suppressed, wherein the image processor includes a bone suppression processing unit configured to suppress the bone structure of the target part based on the plurality of X-ray images generated, a reconstruction processing unit configured to perform, based on the plurality of X-ray images generated, reconstruction for generating the tomographic image, and an adjustment processing unit configured to adjust a suppression degree of the bone structure in the bone-suppressed tomographic image to be generated.

An image processing method according to a second aspect of the present invention includes a step of moving at least one of an X-ray irradiator configured to irradiate a target part of a subject with X-rays and an X-ray detector configured to detect the X-rays with which the subject is irradiated by the X-ray irradiator, and generating a plurality of X-ray images by performing X-ray imaging on the target part of the subject during movement of the at least one of the X-ray irradiator and the X-ray detector; and a step of generating a bone-suppressed tomographic image representing a cross-section of the subject in which a bone structure of the target part is suppressed based on the plurality of X-ray images generated by performing the X-ray imaging, wherein the step of generating the bone-suppressed tomographic image includes a step of suppressing the bone structure of the target part based on the plurality of X-ray images generated, a step of performing, based on the plurality of X-ray images generated, reconstruction for generating the tomographic image, and a step of adjusting a suppression degree of the bone structure in the bone-suppressed tomographic image to be generated.

### Effect of the Invention

In the aforementioned X-ray imaging system according to the first aspect and the aforementioned image processing method according to the second aspect, a bone-suppressed tomographic image representing a cross-section of a subject in which a bone structure of a target part is suppressed is generated. Accordingly, a bone structure (artifact) can be suppressed in a tomographic image by suppressing the bone structure (bone suppression). For this reason, it is possible to suppress appearance of bones in a part in which no bone actually exists in the generated tomographic image. As a result, it is possible to prevent deterioration of visibility of a target part caused by such an artifact of bones in generation of the tomographic image showing the cross-section of the subject. Also, appearance of the bone structure can be more accurately suppressed by adjusting a suppression degree of the bone structure in the generated bone-suppressed tomographic image if the suppression degree of the bone structure is too large or too small. Consequently, it is possible to further prevent deterioration of visibility of the target part in the generated bone-suppressed tomographic image.

### BRIEF DESCRIPTION OF THE DRAWINGS

FIG. 1 is a diagram showing a configuration of an X-ray imaging system according to a first embodiment.

FIG. 2 is a block diagram showing the configuration of the X-ray imaging system according to the first embodiment.

FIG. 3 is a diagram illustrating generation of a bone-suppressed tomographic image in the first embodiment.

FIG. 4 is a view showing a screen of a display in the first embodiment.

FIG. 5 is a diagram illustrating tomosynthesis imaging in the first embodiment.

FIG. 6 is a diagram illustrating image processing for suppressing a bone structure in the first embodiment.

FIG. 7 is a diagram illustrating reconstruction in the first embodiment.

FIG. 8 is a diagram illustrating adjustment of suppression degree of the bone structure in the bone-suppressed tomographic image in the first embodiment.

FIG. 9 is a diagram illustrating an adjustment area in the first embodiment.

FIG. 10 is a flowchart illustrating an image processing method according to the first embodiment.

FIG. 11 is a block diagram showing a configuration of an X-ray imaging system according to a second embodiment.

FIG. 12 is a diagram illustrating adjustment of suppression degree of the bone structure in the bone-suppressed tomographic image in the second embodiment.

FIG. 13 is a diagram showing a configuration of an X-ray imaging system according to a third embodiment.

FIG. 14 is a diagram illustrating adjustment of suppression degree of the bone structure in the bone-suppressed tomographic image in the third embodiment.

### MODES FOR CARRYING OUT THE INVENTION

Embodiments embodying the present invention will be described with reference to the drawings.

### First Embodiment

### Overall Configuration of X-Ray Imaging System

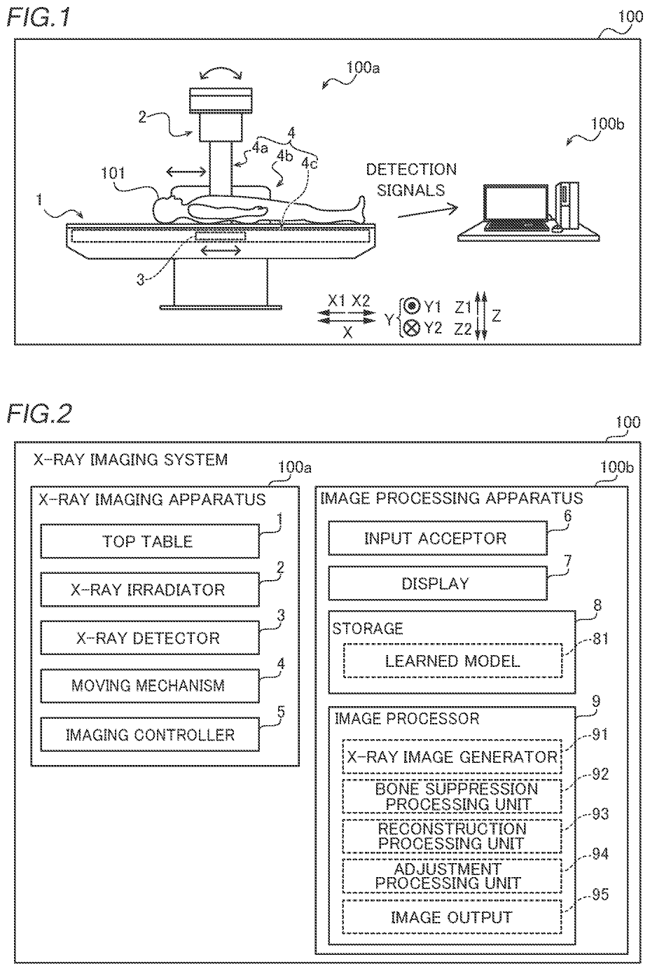

An X-ray imaging system 100 according to a first embodiment of the present invention is now described with reference to FIGS. 1 to 9.

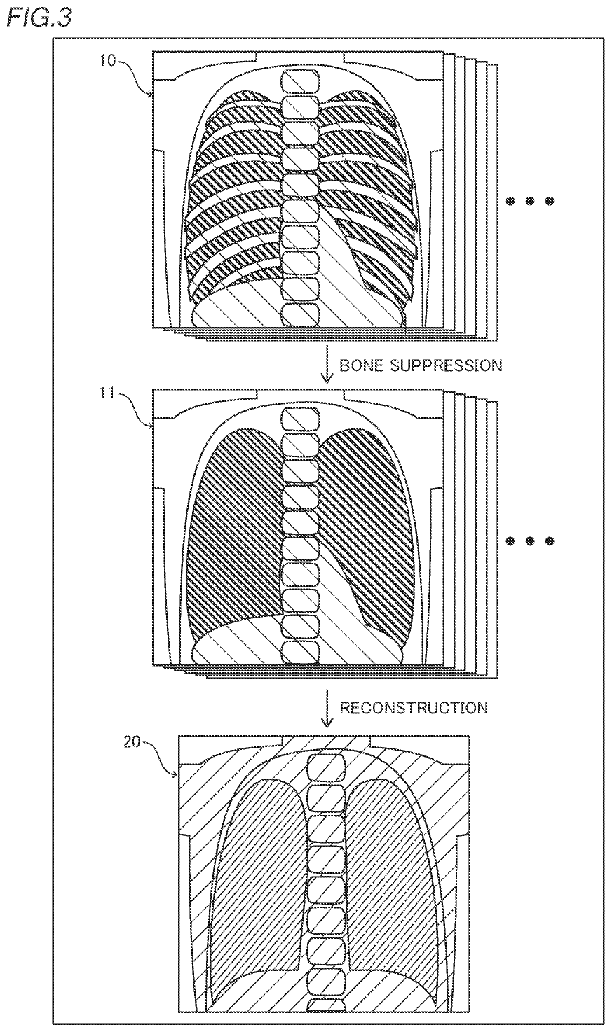

As shown in FIG. 1, the X-ray imaging system 100 is configured to generate a tomographic image showing a cross-section of a subject 101 by performing X-ray imaging (tomosynthesis imaging) on the subject 101. In the first embodiment, a target part of the subject 101 is a thorax (chest and abdomen). The X-ray imaging system 100 is configured to generate a tomographic image for examination of lungs of the subject 101 by performing tomosynthesis imaging on the thorax of the subject 101. The X-ray imaging system 100 is configured to generate a bone-suppressed tomographic image 20 (see FIG. 3), which is a tomographic image in which a bone structure is suppressed in the thorax of the subject 101, and to be able to adjust a suppression degree of a bone structure in the bone-suppressed tomographic image 20 generated.

As shown in FIG. 2, the X-ray imaging system 100 includes an X-ray imaging apparatus 100a, and an image processing apparatus 100b. The X-ray imaging apparatus 100a includes a top table 1, an X-ray irradiator 2, an X-ray detector 3, a moving mechanism 4 and an imaging controller 5. The image processing apparatus 100b includes an input acceptor 6, a display 7, a storage 8, and an image processor 9. For example, the image processing apparatus 100b is a PC (personal computer) used by operators such as doctors.

### Parts of X-Ray Imaging Apparatus

The top table 1 is a bed on which the subject 101 lies. The X-ray irradiator 2 irradiates the chest and the abdomen of the subject 101 who lies on the top table 1 with X rays. The X-ray irradiator 2 includes an X-ray tube configured to irradiate the subject with X rays when a voltage is applied. The X-ray detector 3 is configured to detect the X rays with which the subject 101 is irradiated by the X-ray irradiator 2 and which passes through the subject. The X-ray detector 3 includes a flat panel detector (FPD), for example. The X-ray detector 3 is configured to be able to communicate with the image processor 9, which will be described later, by wireless connection by using wireless LAN, etc., and to output detection signals (image signals) as wireless signals based on the detected X rays to the image processor 9.

As shown in FIG. 1, the moving mechanism 4 is configured to move at least one of the X-ray irradiator 2 and the X-ray detector 3 in accordance with a signal from the imaging controller 5. Specifically, the moving mechanism 4 can change a relative position between the X-ray irradiator 2 and the X-ray detector 3 by moving both the X-ray irradiator 2 and the X-ray detector 3. The moving mechanism 4 includes an irradiator holder 4a, an irradiator mover 4b, and a detector mover 4c. The irradiator holder 4a rotatably holds the X-ray irradiator 2. In other words, the irradiation irradiator holder 4a is configured to be able to change an irradiation angle of the X-ray irradiator 2 in accordance with a signal from the imaging controller 5. The irradiator mover 4b can move the irradiator holder 4a in an X direction in FIG. 1. The detector mover 4c can move the X-ray detector 3 in a direction opposite to the moving direction of the X-ray irradiator 2 in the X direction.

The imaging controller 5 is configured to control the X-ray irradiator 2 and the X-ray detector 3. Specifically, the imaging controller 5 is configured to perform X-ray imaging (tomosynthesis imaging) for generating a plurality of X-ray images 10 (see FIG. 3) of the thorax of the subject 101 while moving the X-ray irradiator 2 and the X-ray detector 3 by controlling the moving mechanism 4. The imaging controller 5 includes a processor such as CPU (Central Processing Unit) or a FPGA (field-programmable gate array. The X-ray images 10 are examples of a “pre-bone-suppression image” in the claims.

### Parts of Image Processing Apparatus

The input acceptor 6 is configured to accept input instructions from an operator such as a doctor. The input acceptor 6 includes a keyboard and a pointing device, such as a computer mouse, for example. In the first embodiment, the input acceptor 6 can accept an input instruction to adjust a suppression degree of the bone structure.

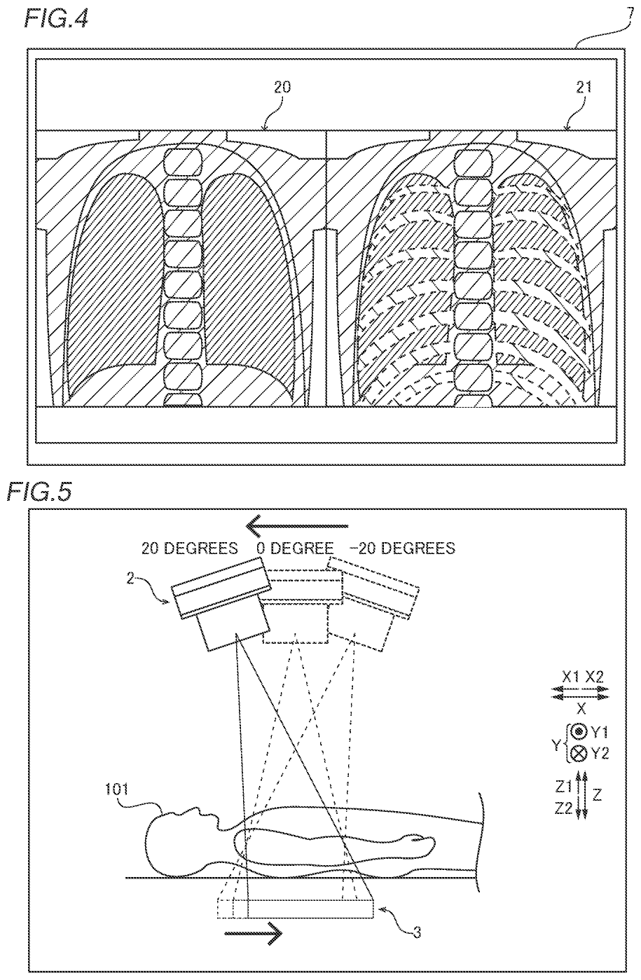

The display 7 is configured to display the bone-suppressed tomographic image 20 and a bone-present tomographic image 21 (see FIG. 4) generated by the image processor 9, which will be described later. The display 7 includes an LCD monitor, for example.

For example, the storage 8 includes a storage device such as a hard disk. The storage 8 is configured to store image data such as X-ray images 10 and the bone-suppressed tomographic image 20 (see FIG. 3) generated by the image processor 9, which will be described later. Also , the storage 8 is configured to store various setting values to be used to operate the X-ray imaging system 100. Also, the storage 8 is configured to store a program to be used to control the X-ray imaging system 100 by using the image processor 9. Also, the storage 8 previously stores a learned model 81, which will be described later.

The image processor 9 is a personal computer including a CPU, a GPU (Graphics Processing Unit), a ROM (Read Only Memory), a RAM (Random Access Memory), etc. In the first embodiment, the image processor 9 is configured to generate a tomographic image showing a cross-section of the subject 101 by suppressing the bone structure (bone suppression) and reconstruction based on the plurality of X-ray images 10 (see FIG. 3) generated by the X-ray imaging (tomosynthesis imaging). Specifically, the image processor 9 is configured to generate a bone-suppressed tomographic image 20 (see FIG. 3), which is a tomographic image in which the bone structure in the thorax is suppressed. In the first embodiment, the bone structure of ribs is suppressed by the bone suppression.

As shown in FIG. 2, the image processor 9 includes an X-ray image generator 91, a bone suppression processing unit 92, a reconstruction processing unit 93, an adjustment processing unit 94, and an image output 95 as functional components. That is, the X-ray image generator 91, the bone suppression processing unit 92, the reconstruction processing unit 93, the adjustment processing unit 94, and the image output 95 are configured to serve as functional blocks as software in the image processor 9 by the image processor 9 as hardware by executing a predetermined control program.

As shown in FIG. 3, the X-ray image generator 91 (image processor 9) is configured to acquire X-ray detection signals (image signals) detected by the X-ray detector 3. The X-ray image generator 91 is configured to generate the X-ray images 10 based on the detection signals acquired. The X-ray images 10 are images generated by capturing images of the thorax of the subject 101 in X-ray imaging.

In the first embodiment, the bone suppression processing unit 92 (image processor 9) is configured to suppress the bone structure (bone suppression) in the thorax based on the plurality of X-ray images 10 generated by tomosynthesis imaging. In the first embodiment, the bone suppression processing unit 92 is configured to suppress the bone structure (ribs) on the plurality of X-ray images 10 before reconstruction is performed by the reconstruction processing unit 93, which will be described later, whereby generating a plurality of bone-suppressed X-ray images 11 in which the ribs are suppressed in the plurality of X-ray images 10. The bone suppression performed by the bone suppression processing unit 92 will be described later. The bone-suppressed X-ray images 11 are examples of a “post-bone-suppression image” in the claims.

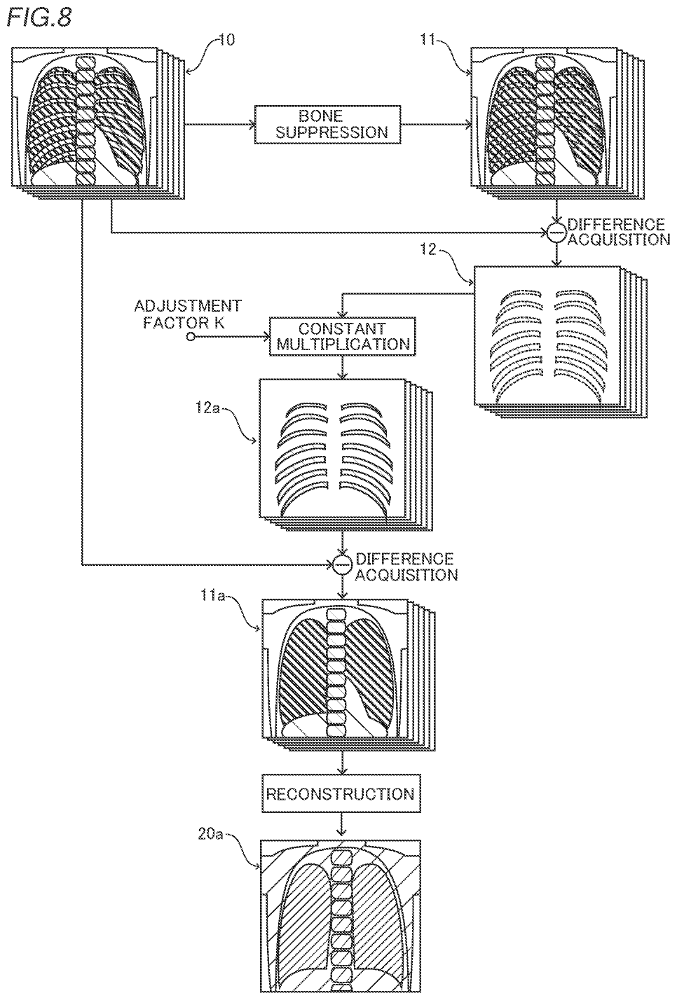

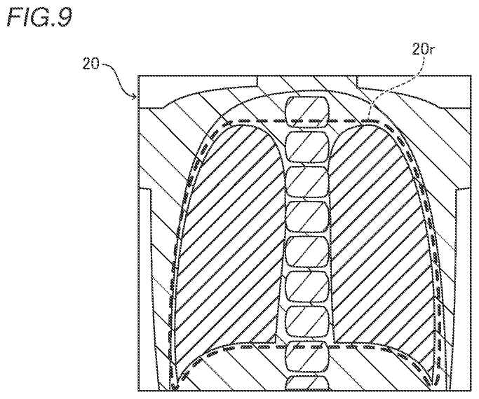

In the first embodiment, the reconstruction processing unit 93 (image processor 9) is configured to perform reconstruction for generating a tomographic image based on the plurality of bone-suppressed X-ray images 11, which are a plurality of X-ray images 10 in which the ribs are suppressed, whereby generating a bone-suppressed tomographic image 20. Also, in order to adjust the suppression degree of the bone structure in the bone-suppressed tomographic image 20 generated, the reconstruction processing unit 93 is configured to perform the reconstruction based on a plurality of adjusted bone-suppressed X-ray images 11a (see FIG. 8), which are X-ray images 10 whose suppression degree of the bone structure is adjusted by the adjustment processing unit 94 described later, generating an adjusted bone-suppressed tomographic image 20a, which is the bone-suppressed tomographic image 20 whose suppression degree of the bone structure is adjusted. Also, the reconstruction processing unit 93 is configured to perform the reconstruction on the plurality of X-ray images 10 before the suppression of the bone structure is performed whereby generating the bone-present tomographic image 21 (see FIG. 4). The bone-present tomographic image 21 is an image (image including the bone structure of the ribs) representing a cross-section of the subject 101 including an artifact of the ribs in lung parts in which no rib exists. The reconstruction performed by the reconstruction processing unit 93 will be described later.

The adjustment processing unit 94 (image processor 9) is configured to adjust the suppression degree of the bone structure in the bone-suppressed tomographic image 20 generated. In the first embodiment, the adjustment processing unit 94 is configured to adjust the suppression degree of the bone structure in the plurality of bone-suppressed X-ray images 11 generated by the bone suppression processing unit 92 whereby generating the plurality of adjusted bone-suppressed X-ray images 11a (see FIG. 8). The adjustment of the suppression degree of the bone structure will be described later.

As shown in FIG. 4, the image output 95 (image processor 9) is configured output the bone-suppressed tomographic image 20 generated by the reconstruction processing unit 93. Specifically, the image output 95 is configured to direct the display 7 to display the bone-suppressed tomographic image 20. Also, the image output 95 is configured to direct the display 7 to display the bone-present tomographic image 21 generated by the reconstruction processing unit 93. For example, the image output 95 is configured to direct the display 7 to display the bone-suppressed tomographic image 20 and the bone-present tomographic image 21 adjacent to each other.

### Tomosynthesis Imaging

As shown in FIG. 5, the X-ray imaging apparatus 100a is an apparatus for tomosynthesis imaging. For example, the X-ray imaging apparatus 100a moves the X-ray irradiator 2 in an X1 direction while changing an irradiation angle of the X-ray irradiator, and moves the X-ray detector 3 in an X2 direction to capture X-ray images of the subject. Specifically, the imaging controller 5 controls movement of the moving mechanism 4 so as to move the X-ray irradiator 2 from a position of −20 degrees on an X2-direction side to a position of +20 degrees on an X1-direction side with respect to the subject 101 as a vertical direction (Z direction in FIG. 1) defined as a reference (zero degree). The imaging controller 5 captures an X-ray image of the subject at every one-degree movement of the X-ray irradiator 2 from the position of −20 degrees to the position of +20 degrees whereby capturing 41 X-ray images 10 of the subject. The imaging controller 5 additionally rotates the X-ray irradiator 2 one degree by one degree about an axis of a Y direction corresponding to the movement of the X-ray irradiator 2 in the X1 direction. Also, the imaging controller 5 is configured to move the X-ray detector 3 in the X2 direction corresponding to the movement of the X-ray irradiator 2 in the X1 direction.

As a result, the X-ray imaging apparatus 100a captures 41 X-ray images 10 at different positions (irradiation angles) from −20 degrees to +20 degrees in X-ray imaging. The X-ray image generator 91 (image processor 9) of the image processing apparatus 100b generates 41 X-ray images 10 based on the detection signals (image signals) of 41 images captured (detected) by the X-ray imaging apparatus 100a.

### Bone Suppression

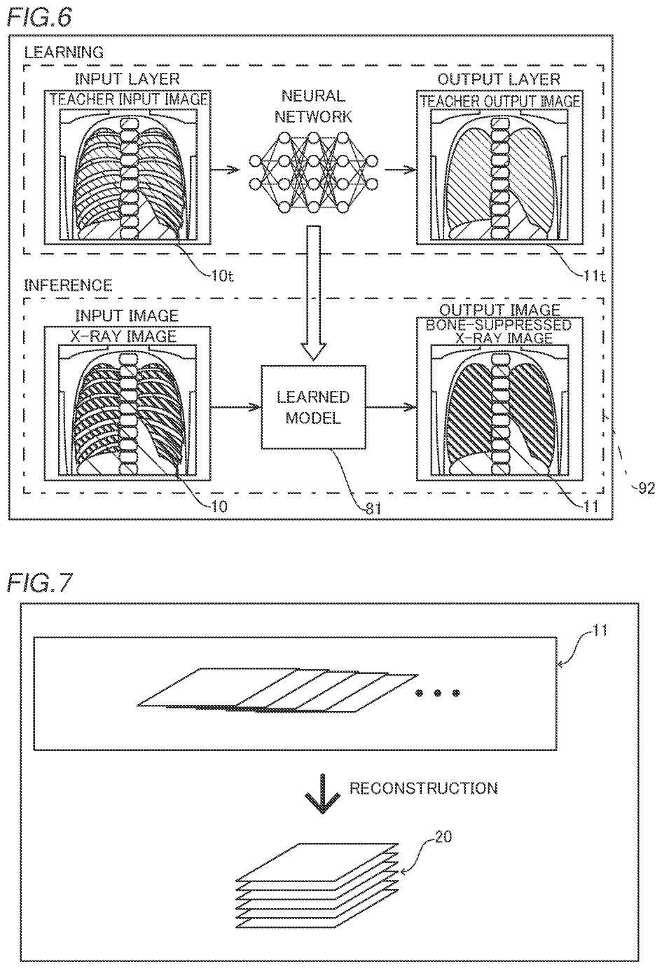

As shown in FIG. 6, the bone suppression processing unit 92 (image processor 9) is configured to suppress the bone suppression (bone suppression), which is processing for suppressing the bone structure, on each of the 41 X-ray images 10. Specifically, the bone suppression processing unit 92 (image processor 9) is configured to perform image processing by using a learned model 81 that is produced by machine learning to suppress the bone structure (perform bone suppression) in the first embodiment. More specifically, the bone suppression processing unit 92 performs image processing based on the learned model 81 produced by machine learning to suppress (remove) the bone structure of the ribs in the thorax of the subject 101 from the X-ray images 10.

The learned model 81 is produced by machine learning using deep learning to generate bone-suppressed X-ray images 11 in which ribs are suppressed in the X-ray images 10. The learned model 81 is previously produced by a training apparatus provided separately from the image processing apparatus 100b, and stored in the storage 8. The training device produces the learned model 81 by machine learning using teacher data (training set) including a number of teacher input images 10t and a number of teacher output images 11t. The teacher input images 10t are generated to simulate X-ray images 10 of thoraxes of the subjects 101. The teacher output images 11t are images in which ribs are suppressed in (removed from) the teacher input images 10t. The teacher input images 10t and the teacher output images 11t are generated to meet conditions similar to the input X-ray images 10 used as inputs in inference using the learned model 81.

For example, the learned model 81 is produced based on Fully Convolution Network (FCN). The learned model 81 is produced by converting pixels that are estimated as ribs (replacing pixel values of a bone structure (bone) with pixel values of soft tissues such as muscles) in pixels of the thorax of the X-ray image 10 as an input to learn image conversion (image processing) that suppresses an image of ribs in the X-ray image 10.

The bone suppression processing unit 92 (image processor 9) is configured to generate 41 bone-suppressed X-ray images 11 by performing bone suppression using the learned model 81 on each of the 41 X-ray images 10.

### Reconstruction

As shown in FIG. 7, the reconstruction processing unit 93 (image processor 9) of the image processing apparatus 100b is configured to reconstruct 41 bone-suppressed X-ray images 11, and to be able to generate a bone-suppressed tomographic image 20 of the subject 101 at an arbitrary height (thickness) from cross-sections parallel to a detection plane of the X-ray detector 3 (cross-sections parallel to the moving direction of the X-ray irradiator 2). In the first embodiment, the bone-suppressed tomographic image 20 is an image of the cross-section of the subject 101 parallel to an X-Y plane in FIG. 1 at an arbitrary height (thickness) in the Z direction. The reconstruction performed by the reconstruction processing unit 93 includes iterative reconstruction (IR), filtered back projection (FBP), shift-and-add, etc. For example, T-SMART (Tomosynthesis Shimadzu Artifact Reduction Technology), which is a reconstruction method to which the iterative reconstruction is applied, etc. can be used for reconstruction performed by the reconstruction processing unit 93.

### Adjustment of Suppression Degree

Here, in some cases, the bone structure is suppressed too much or too little in the bone-suppressed tomographic image 20 generated by the reconstruction processing unit 93. For example, if the suppression of bone structure is insufficient, the bone structure (artifact) remains in the bone-suppressed tomographic image 20. On the other hand, if the suppression of the bone structure is too much, appearance of the bone-suppressed tomographic image 20 will be unnatural, or parts other than the bone structure (such as pulmonary vessels) will be suppressed. The X-ray imaging system 100 according to the first embodiment is configured to be able to adjust the suppression degree of the bone structure in the bone-suppressed tomographic image 20 in accordance with an instruction input by an operator such as a doctor who sees the bone-suppressed tomographic image 20 displayed on the display 7, for example.

As shown in FIG. 8, in the first embodiment, the adjustment processing unit 94 (image processor 9) is configured to adjust the suppression degree of the bone structure in the bone-suppressed tomographic image 20 based on the plurality of X-ray images 10 (pre-bone-suppression images) before the bone structure is suppressed by the bone suppression processing unit 92, and the plurality of bone-suppressed X-ray images 11 (post-bone-suppression images) after the bone structure is suppressed by the bone suppression processing unit 92.

Specifically, the adjustment processing unit 94 is configured to adjust the suppression degree of the bone structure based on the plurality of bone-suppressed X-ray images 11, which are results of suppression by the bone suppression processing unit 92. In other words, the adjustment processing unit 94 is configured to adjust the suppression degree of the bone structure in the bone-suppressed tomographic image 20 by adjusting the suppression degree of the bone structure in the plurality of bone-suppressed X-ray images 11 before the reconstruction is performed by the reconstruction processing unit 93.

Specifically, the adjustment processing unit 94 acquires differences between the plurality of X-ray images 10 before the bone structure is suppressed and the plurality of bone-suppressed X-ray images 11 after the bone structure is suppressed to generate a plurality of bone-extracted images 12. That is, the adjustment processing unit 94 generates the bone-extracted images 12 by acquiring differences between the X-ray images 10 before bone suppression is performed by the bone suppression processing unit 92 and the bone-suppressed X-ray images 11 after bone suppression is performed by the bone suppression processing unit 92. The adjustment processing unit 94 is configured to acquire differences between one X-ray image 10 before the bone structure is suppressed in the 41 X-ray images 10 and one bone-suppressed X-ray image 11 after the bone structure is suppressed corresponding the one X-ray image 10 in the 41 bone-suppressed X-ray images 11. Each bone-extracted image 12 is an image in which the bone structure (ribs) is only extracted from the target part of the subject 101.

The adjustment processing unit 94 (image processor 9) performs constant multiplication on the bone-extracted image 12 generated Specifically, in the first embodiment, the adjustment processing unit 94 is configured to adjust the suppression degree of the bone structure in the bone-suppressed tomographic image 20 based on a predetermined adjustment factor K. The adjustment processing unit 94 generates the adjusted bone-extracted image 12a by multiplying a pixel value of each pixel included in the generated bone-extracted image 12 by the adjustment factor K. More specifically, the adjustment processing unit 94 generates a plurality of adjusted bone-extracted images 12a by performing constant multiplication on the plurality of bone-extracted images 12. Subsequently, the adjustment processing unit 94 generates the adjusted bone-suppressed X-ray images 11a by acquiring differences between the plurality of X-ray images 10 and the plurality of adjusted bone-extracted images 12a. The adjusted bone-suppressed X-ray images 11a are bone-suppressed X-ray images 11 in which the suppression degree of the bone structure is changed in accordance with the adjustment factor K to adjust the suppression degree of the bone structure in the bone-suppressed tomographic image 20. Subsequently, the reconstruction processing unit 93 performs the reconstruction on the plurality of adjusted bone-suppressed X-ray images 11a whereby generating the adjusted bone-suppressed tomographic image 20a, which is the bone-suppressed tomographic image 20 whose suppression degree of the bone structure is adjusted, similar to the bone-suppressed X-ray images 11. The image output 95 is configured to direct the display 7 to display the generated adjusted bone-suppressed tomographic image 20a and the bone-present tomographic image 21 adjacent to each other similar to the bone-suppressed tomographic image 20 before the suppression degree of bone structure is adjusted.

In the aforementioned adjustment of the suppression degree of the bone structure by the adjustment processing unit 94, where each pixel value of each generated X-ray image 10 is P, each pixel value of each bone-suppressed X-ray image 11 is S, and each pixel value of each adjusted bone-suppressed X-ray image 11a is Sa, because the bone-extracted images 12 are differences between the X-ray images 10 and the bone-suppressed X-ray images 11, a pixel value Sa of each adjusted bone-suppressed X-ray image 11a is represented by Equation (1).

$$Sa = P - K \cdot ( P - S ) = ( 1 - K ) \cdot P + K \cdot S \qquad ( 1 )$$

For this reason, similar adjusted bone-suppressed X-ray images 11a can be acquired even if calculation using the adjustment factor K is performed on the X-ray images 10 and the bone-suppressed X-ray images 11 without generating the bone-extracted image 12.

In a case in which the adjustment factor K is greater than 1, the bone structure in the adjusted bone-extracted images 12a becomes higher (greater in intensity), and as a result a suppression intensity of the bone structure in the adjusted bone-suppressed X-ray images 11a becomes greater. Accordingly, if the adjustment factor K is greater than 1, the adjustment processing unit 94 adjusts the suppression degree of the bone structure to a greater degree in the bone-suppressed tomographic image 20. In a case in which the adjustment factor K is smaller than 1, the bone structure in the adjusted bone-extracted images 12a becomes lower (smaller in intensity), and as a result a suppression intensity of the bone structure in the adjusted bone-suppressed X-ray images 11a becomes smaller. Accordingly, if the adjustment factor K is smaller than 1, the adjustment processing unit 94 adjusts the suppression degree of the bone structure to a smaller degree in the bone-suppressed tomographic image 20. In a case in which the adjustment factor K is set 0, the adjusted bone-suppressed tomographic image 20a is generated without suppressing the bone structure. In this case, the generated adjusted bone-suppressed tomographic image 20a becomes similar to the bone-present tomographic image 21.

In the first embodiment, the adjustment processing unit 94 (image processor 9) is configured to change the adjustment factor K in accordance with on an input instruction accepted by the input acceptor 6. For example, an operator such as a doctor adjusts the suppression degree of the bone structure by inputting an instruction to change the adjustment factor K in magnitude through the input acceptor 6 while seeing the unadjusted bone-suppressed tomographic image 20 displayed on the display 7. The adjustment processing unit 94 is configured to set the adjustment factor K in accordance with the input instruction accepted, and to adjust the suppression degree of the bone structure in accordance with the set adjustment factor K.

As shown in FIG. 9, in the first embodiment, the adjustment processing unit 94 (image processor 9) is configured be able to adjust the suppression degree of the bone structure in each pixel of the bone-suppressed tomographic image 20 generated. Specifically, the adjustment processing unit 94 acquires an adjustment area 20r, which is an area whose suppression degree of the bone structure is adjusted in accordance with the instruction input through the input acceptor 6. The adjustment processing unit 94 is configured to adjust (change) the suppression degree of the bone structure for each pixel in the area specified as the adjustment area 20r of the bone-suppressed tomographic image 20.

For example, an area corresponding to a lung field in the bone-suppressed tomographic image 20 is specified (selected) as the adjustment area 20r based on the instruction input by the operator such as doctor through the input acceptor 6. In addition, the adjustment factor K is set to adjust (change) the suppression degree of the bone structure in the area of the lung field (adjustment area 20r) in accordance with the instruction input through the input acceptor 6. The adjustment processing unit 94 performs the aforementioned calculation for adjusting the suppression degree of the bone structure in accordance with the set adjustment factor K only for pixels that are included in the adjustment area 20r specified. As a result, the adjustment processing unit 94 generates the bone-suppressed tomographic image 20 in which the suppression degree of the bone structure is adjusted only for the pixels that are included in the adjustment area 20r. No suppression of the bone structure can be performed on (the adjustment factor K can be set 0 for) pixels that are not included in the adjustment area 20r.

### Image Processing Method of First Embodiment

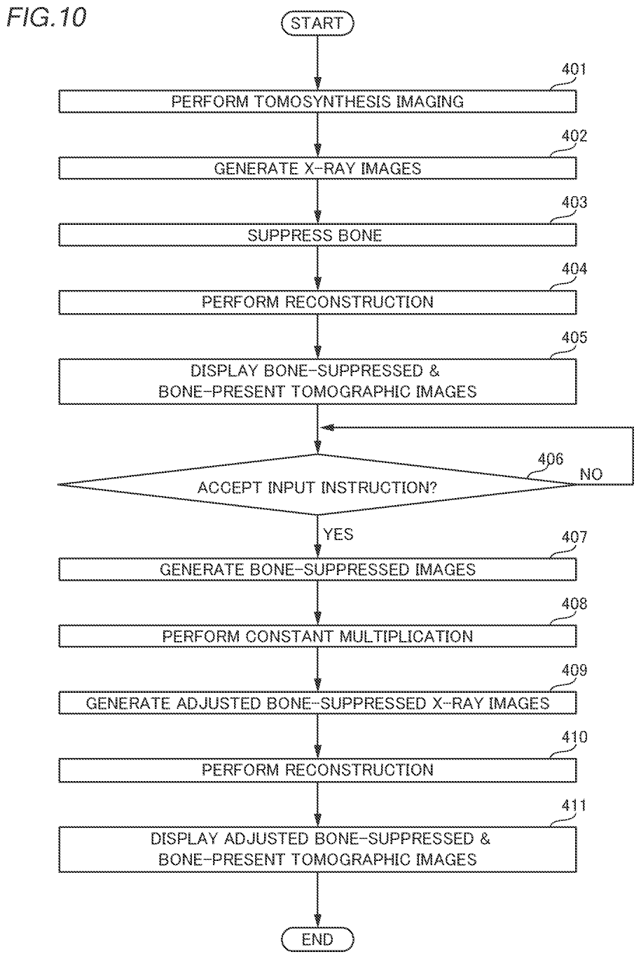

A control flow of an image processing method according to the first embodiment is now described with reference to FIG. 10. Step 401 presents control processing performed by the imaging controller 5 of the X-ray imaging apparatus 100a, and steps 402 to 411 represent control processing performed by the image processor 9 of the image processing apparatus 100b.

Firstly, in step 401, X-ray imaging (tomosynthesis imaging) is performed on the thorax (target part) of the subject 101 while moving the X-ray irradiator 2 and the X-ray detector 3.

Subsequently, in step 402, a plurality of (41) X-ray images 10 are generated based on the detection signals (image signals) acquired in the tomosynthesis imaging performed in step 401.

Subsequently, in step 403, the bone structure of the ribs is suppressed based on the generated 41 X-ray images 10 in the thorax. Specifically, 41 bone-suppressed X-ray images 11 in which the bone structure of the ribs is suppressed are generated by suppressing the bone structure on the X-ray images 10.

Specifically, in step 404, reconstruction for generating a tomographic image is performed based on the 41 bone-suppressed X-ray images 11, which are X-ray images 10 in which the bone structure is suppressed. Specifically, the bone-suppressed tomographic image 20, which is a cross-sectional image of the subject 101 in which the bone structure of the ribs is suppressed, is generated by the reconstruction of the bone-suppressed X-ray images 11. Also, a bone-present tomographic image 21 is generated by reconstruction of the 41 X-ray images 10.

Specifically, in step 405, the display 7 displays the bone-suppressed tomographic image 20 generated and the bone-present tomographic image 21 generated. Specifically, the bone-suppressed tomographic image 20 and the bone-present tomographic image 21 are displayed side by side on the display 7.

Subsequently, in step 406, it is determined whether an input instruction to adjust the suppression degree of the bone structure in the bone-suppressed tomographic image 20 is accepted by the input acceptor 6. Specifically, it is determined whether an input instruction to change the adjustment factor K to adjust the suppression degree of the bone structure is accepted. If it is determined that the input instruction to adjust the suppression degree of the bone structure is accepted, the procedure goes to step 407. If it is not determined that the input instruction to adjust the suppression degree of the bone structure is accepted, the procedure repeats step 406.

In step 407, differences between the plurality of (41) X-ray images 10 generated in step 402 and the plurality of (41) bone-suppressed X-ray images 11 generated in step 403 are acquired. Specifically, a plurality of bone-extracted images 12 are generated by acquiring the differences between the plurality of X-ray images 10 and the plurality of bone-suppressed X-ray images 11.

Subsequently, in step 408, constant multiplication is performed on the bone-extracted images 12 generated. Specifically, the plurality of adjusted bone-extracted images 12a are generated by multiplying a pixel value of each pixel included in the generated plurality of bone-extracted images 12 by the input adjustment factor K.

Subsequently, in step 409, differences between the plurality of (41) X-ray images 10 generated in step 402 and the plurality of (41) adjusted bone-extracted images 12a generated in step 408 are acquired. Specifically, a plurality of adjusted bone-suppressed X-ray images 11a are generated by acquiring differences between the plurality of X-ray images 10 and the plurality of adjusted bone-extracted images 12a.

Subsequently, in step 410, reconstruction processing is performed on the plurality of adjusted bone-suppressed X-ray images 11a. Specifically, the adjusted bone-suppressed tomographic image 20a, which is a bone-suppressed tomographic image 20 whose the suppression degree of the bone structure of the ribs is adjusted, is generated by performing the reconstruction on the adjusted bone-suppressed X-ray images 11a. That is, the suppression of the bone structure in step 403 and the reconstruction in step 404 are performed based on the plurality of X-ray images 10 generated in step 402, and the adjusted bone-suppressed tomographic image 20a whose suppression degree of the bone structure is adjusted is generated by processes for adjusting the suppression degree of the bone structure in steps 407 to 410.

Subsequently, in step 411, the adjusted bone-suppressed tomographic image 20a generated in step 410 and the bone-present tomographic image 21 generated in step 404 are displayed on the display 7. Specifically, the bone-suppressed tomographic image 20 and the bone-present tomographic image 21 are displayed adjacent to each other. on the display 7.

If an input instruction to specify an adjustment area 20r is accepted together with in step 406 along with the input instruction to change the adjustment factor K, the processes for adjusting the degree suppression of the bone structure in steps 407 to 410 are performed for pixels that are included only in the specified adjustment area 20r of the bone-suppressed tomographic image 20.

### Advantages of First Embodiment

In the first embodiment, the following advantages are obtained.

In the X-ray imaging system 100 according to the first embodiment, as discussed above, the bone-suppressed tomographic image 20, which is a tomographic image representing a cross-section of the subject 101 in which the bone structure (ribs) in the target part (thorax) is suppressed, is generated. Accordingly, the bone structure (artifact) can be suppressed in the tomographic image by suppressing the bone structure (bone suppression). For this reason, it is possible to suppress appearance of ribs in a part in which no rib (no bone) actually exists in the generated tomographic image. As a result, it is possible to prevent deterioration of visibility of the target part (thorax) caused by such an artifact of bones (ribs) in generation of the tomographic image showing the cross-section of the subject 101. Also, appearance of the bone structure can be more accurately suppressed by adjusting a suppression degree of the bone structure in the generated bone-suppressed tomographic image 20 if the suppression degree of the bone structure is too large or too small. Consequently, it is possible to further prevent deterioration of visibility of the target part in the generated bone-suppressed tomographic image 20 (adjusted bone-suppressed tomographic image 20a).

In addition, following additional advantages can be obtained by the aforementioned first embodiment added with configurations discussed below.

That is, in the first embodiment, as discussed above, the bone suppression processing unit 92 (image processor 9) is configured to suppress the bone structure on each of the plurality of X-ray images 10 before the reconstruction is performed, and the adjustment processing unit 94 (image processor 9) is configured to adjust the suppression degree of the bone structure in the bone-suppressed tomographic image 20 generated by performing the adjustment of the suppression degree of the bone structure based on the bone-suppressed X-ray images 11 (post-suppression images), which are results of suppression by the bone suppression processing unit 92. According to this configuration, because the plurality of X-ray images 10 before the reconstruction is performed are images captured at predetermined irradiation positions (irradiation angles), an image of ribs can be more easily suppressed as compared with a case in which the ribs are suppressed in the tomographic image after reconstruction. Consequently, it is possible to easily prevent deterioration of visibility of the thorax because the bone-suppressive tomographic image 20 can be easily generated. In addition, because the suppression degree of the bone structure is suppressed based on the plurality of bone-suppressed X-ray images 11, which are results of suppression by the bone suppression processing unit 92, the suppression degree of the bone structure can be adjusted by image processing. Consequently, because the suppression degree of the bone structure can be adjusted without changing (adjusting) parameters of calculation for suppression of the bone structure itself, it is possible to easily adjust the suppression degree of the bone structure.

In the first embodiment, as discussed above, the adjustment processing unit 94 (image processor 9) is configured to adjust the suppression degree of the bone structure in the bone-suppressed tomographic image 20 based on the X-ray images 10 (pre-bone-suppression images) before the bone structure is suppressed by the bone suppression processing unit 92 (image processor 9), and the bone-suppressed X-ray images 11 (post-bone-suppression images) after the bone structure is suppressed by the bone suppression processing unit 92. According to this configuration, the suppression degree of the bone structure by the bone suppression processing unit 92 can be easily adjusted by using the X-ray images 10 before the bone structure is suppressed and the bone-suppressed X-ray images 11 after the bone structure is suppressed. Consequently, the suppression degree of the bone structure can be easily adjusted in the bone-suppressed tomographic image 20 generated.

In the first embodiment, as discussed above, an input acceptor 6 configured to accept an input instruction to adjust the suppression degree of the bone structure is further provided; and the adjustment processing unit 94 (image processor 9) is configured to adjust the suppression degree of the bone structure in the bone-suppressed tomographic image 20 based on a predetermined adjustment factor K and to set the adjustment factor K based on the input instruction accepted by the input acceptor 6. According to this configuration, because the adjustment factor K can be changed (adjusted) based on input instruction accepted by the input acceptor 6, the suppression degree of the bone structure can be more easily adjusted in the bone-suppressed tomographic image 20 generated. Accordingly, because operators such as doctors can easily see the adjusted bone-suppressed tomographic image 20a while adjusting the suppression degree of the bone structure, they can easily recognize change resulting from the adjusted suppression degree of the bone structure. Consequently, because they can easily recognize an area of the image in which the bone structure is suppressed, it is possible to improve visibility of a lesion part. Also, for example, if an operator such as a doctor who sees the generated bone-suppressed tomographic image 20 has a feeling that something is wrong in the bone-suppressed tomographic image 20 in which the bone structure is suppressed, the operator can reduce the suppression of the bone structure in the bone-suppressed tomographic image 20 (can make an effect of the suppression weaker) by decreasing the adjustment factor K. Because the bone-suppressed tomographic image 20 can be adjusted close to a tomographic image including the bone structure by decreasing the adjustment factor K, even if the operator such as doctor has a feeling that something is wrong in the bone-suppressed tomographic image 20 in which the bone structure is suppressed, the operator can adjust the bone-suppressed tomographic image 20 to an image that provide good visibility for the operator such as doctor.

In the first embodiment, as discussed above, the adjustment processing unit 94 (image processor 9) is configured be able to adjust the suppression degree of the bone structure in each pixel of the bone-suppressed tomographic image 20 generated. According to this configuration, the suppression of the bone structure can be not entirely but locally adjusted in the bone-suppressed tomographic image 20, in other words, can be adjusted only in a part (some of areas) of the bone-suppressed tomographic image. Accordingly, it is possible to suppress only the bone structure while preventing that suppression of the bone structure is performed on areas other than the bone structure. Because suppression of the bone structure can be performed only on a required area while while preventing that suppression of the bone structure is performed on non-required areas, a lesion part can be more accurately examined in the thorax of the subject 101 by seeing the bone-suppressed tomographic image 20 (adjusted bone-suppressed tomographic image 20a) generated.

In the first embodiment, as discussed above, the bone suppression processing unit 92 (image processor 9) is configured to suppress the bone structure on each of the plurality of X-ray images 10 generated; the adjustment processing unit 94 (image processor 9) is configured to adjust the suppression degree of the bone structure in the plurality of the bone-suppressed X-ray images 11 in which the bone structure is suppressed by the bone suppression processing unit 92; and the reconstruction processing unit 93 (image processor 9) is configured to generate the adjusted bone-suppressed tomographic image 20a in which the suppression degree of the bone structure is adjusted by performing the reconstruction based on the plurality of adjusted bone-suppressed X-ray images 11a in which the suppression degree of the bone structure is adjusted by the adjustment processing unit 94. According to this configuration, because suppression of the bone structure is performed not on the reconstructed tomographic image but on the X-ray images 10, the bone structure can be suppressed in the plurality of X-ray images 10 whose appearances are relatively constant as compared with a tomographic image whose appearance changes depending on a height (thickness) position of the cross-section of the subject 101. Because the bone structure can be accurately suppressed, it is possible to accurately improve visibility of a lesion part in the bone-suppressed tomographic image 20 generated and the adjusted bone-suppressed tomographic image 20a.

In the first embodiment, as discussed above, the reconstruction processing unit 93 (image processor 9) is configured to generate a bone-present tomographic image 21, which is a tomographic image including the bone structure of the thorax (target part), by performing the reconstruction on the plurality of X-ray images 10; and a display 7 configured to display the bone-present tomographic image 21 including the bone structure, and the bone-suppressed tomographic image 20 in which the bone structure is suppressed is further provided. According to this configuration, an operator such as a doctor can easily compare the target part (thorax) of the subject 101 with the bone structure (ribs) being suppressed to the target part (thorax) of the subject 101 that includes the ribs by seeing the display 7 displaying the bone-present tomographic image 21 and the bone-suppressed tomographic image 20. Consequently, a lesion part in the thorax of the subject 101 can be easily examined by comparing appearances between the bone-suppressed tomographic image 20 and the bone-present tomographic image 21 displayed on the display 7.

In the first embodiment, as discussed above, the target part includes the thorax (chest and abdomen) of the subject 101; and the image processor 9 is configured to generate the bone-suppressed tomographic image 20 in which the bone structure including the rib is suppressed in the thorax. Here, in tomosynthesis imaging of the thorax, ribs, which enclose the lungs, sometimes appear in a cross-section of the lungs (rib artifacts sometimes appear). Accordingly, such a rib artifact in the cross-section of the lungs may reduce visibility of a lesion part in the lungs, and make examination (diagnosis) of the lungs difficult. Contrary to this, in the first embodiment, the target part includes the thorax of the subject 101; and the image processor 9 is configured to generate the bone-suppressed tomographic image 20 in which the bone part including the ribs is suppressed in the thorax. According to this configuration, because the bone-suppressed tomographic image 20 in which the ribs are suppressed in a tomographic image including the lungs in the thorax can be generated, it is possible to prevent that the artifact of the ribs deteriorates of visibility of a lesion part in the lungs.

In the first embodiment, as discussed above, the bone suppression processing unit 92 (image processor 9) is configured to suppress the ribs (bone structure) of the target part by performing image processing by using a learned model 81 that is produced by machine learning to suppress the bone structure (ribs). According to this configuration, a bone-suppressed tomographic image 20 in which an image of ribs is accurately suppressed can be generated by using the learned model 81 produced by machine learning. Also, because the bone-suppressed tomographic image 20 can be generated by using the learned model 81, the bone-suppressed tomographic image 20 can be generated by changing an image processing software configuration of a conventional tomosynthesis imaging apparatus to a software configuration image processing software configuration that is configured to generate the bone-suppressed tomographic image. For this reason, the bone-suppressed tomographic image 20 can be generated without changing both an imaging method and an apparatus configuration of the conventional tomosynthesis imaging apparatus. Because the bone-suppressed tomographic image 20 in which the bone structure is accurately suppressed can be generated by using the learned model 81 without making the imaging apparatus complicated to generate the bone-suppressed tomographic image 20 in which the bone structure is suppressed, it is possible to accurately suppress the artifact of ribs and to more easily prevent deterioration of visibility of thorax caused by the artifact of ribs.

### Advantages of Image Processing Method of First Embodiment

In the image processing method according to the first embodiment, the following advantages are obtained.

In the image processing method according to the first embodiment, according to the aforementioned configuration, the bone-suppressed tomographic image 20, which is a tomographic image representing a cross-section of the subject 101 in which the bone structure (ribs) in the target part (thorax) is suppressed, is generated. Accordingly, the bone structure (artifact) can be suppressed in the tomographic image by suppressing the bone structure (bone suppression). For this reason, it is possible to suppress appearance of ribs in a part in which no rib (no bone) actually exists in the generated tomographic image. As a result, it is possible to provide an image processing method capable of preventing deterioration of visibility of the target part (thorax) caused by such an artifact of bones (ribs) in generation of the tomographic image showing the cross-section of the subject 101. Also, appearance of the bone structure can be more accurately suppressed by adjusting a suppression degree of the bone structure in the generated bone-suppressed tomographic image 20 if the suppression degree of the bone structure is too large or too small. Consequently, it is possible to provide an image processing method capable of further preventing deterioration of visibility of the target part in the generated bone-suppressed tomographic image 20 (adjusted bone-suppressed tomographic image 20a).

### Second Embodiment

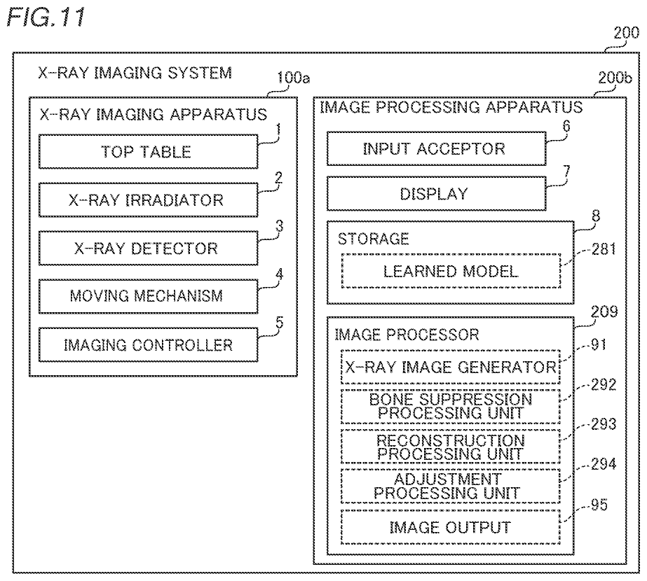

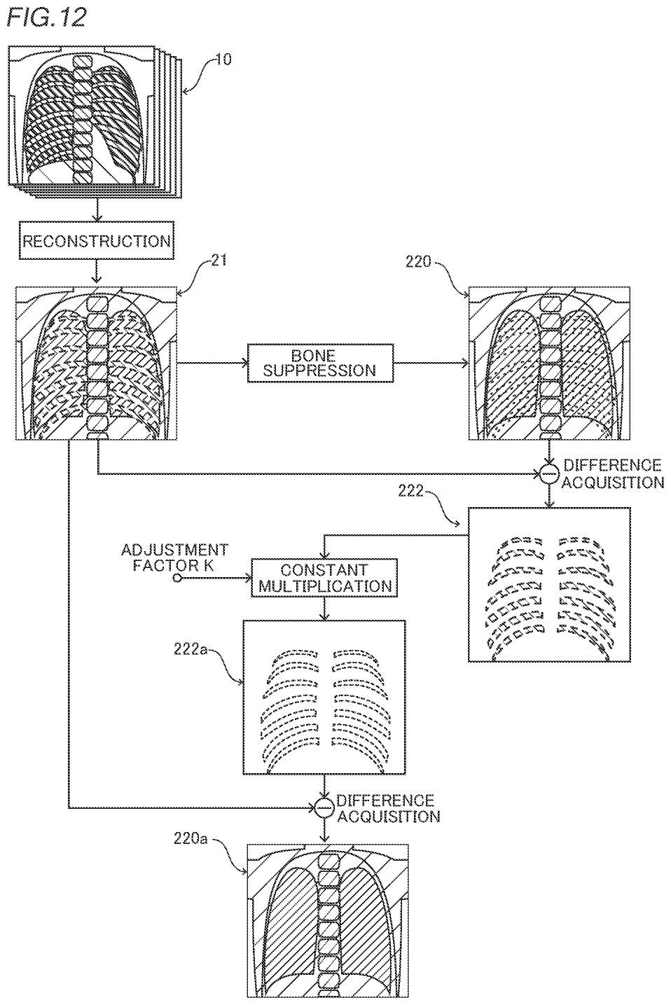

An X-ray imaging system 200 according to a second embodiment is now described with reference to FIGS. 11 and 12. Dissimilar to the X-ray imaging system according to the first embodiment, which is configured to suppress the bone structure (ribs) in a plurality of X-ray images 10 before the reconstruction is performed, the X-ray imaging system according to the second embodiment is configured to suppress the bone structure in a bone-present tomographic image 21, which is a tomographic image after reconstruction is performed. The same configurations in the second embodiment as those of the first embodiment are denoted by the same reference numerals, and their description is omitted.

As shown in FIG. 11, the X-ray imaging system 200 according to the second embodiment includes an X-ray imaging apparatus 100a, and an image processing apparatus 200b. The image processing apparatus 200b includes an image processor 209. Similar to the image processor 9 in the first embodiment, the image processor 209 is configured to generate a bone-suppressed tomographic image 220 based on X-ray images 10. In the second embodiment, the image processor 209 is configured to generate the bone-suppressed tomographic image 220 by suppressing the bone structure (ribs) in the bone-present tomographic image 21 after the reconstruction is performed. Also, the image processor 209 includes an X-ray image generator 91, a bone suppression processing unit 292, a reconstruction processing unit 293, an adjustment processing unit 294, and an image output 95 as functional components.

As shown in FIG. 12, the X-ray image generator 91 (image processor 209) generates 41 X-ray images 10 based on the detection signals (image signals) of images captured (detected) by X-ray imaging (tomosynthesis imaging) by using the X-ray imaging apparatus 100a similar to the first embodiment.

The reconstruction processing unit 293 (image processor 209) is configured to perform reconstruction for generating a bone-present tomographic image 21, which is a tomographic image including a bone part (bone structure), by performing the reconstruction on the plurality of X-ray images 10 captured before the bone structure (rib) is suppressed (before bone suppression). Processes of the reconstruction are similar to the first embodiment.

In the second embodiment, the bone suppression processing unit 292 (image processor 209) is configured to generate the bone-suppressed tomographic image 220 by suppressing the bone structure (ribs) in (performing bone suppression on) the bone-present tomographic image 21, which is generated by the reconstruction processing unit 293, after the reconstruction is performed. For example, the bone suppression processing unit 292 generates a bone-suppressed tomographic image 220 of ribs from a bone-present tomographic image 21, which is a tomographic image including ribs, based on a learned model 281 (see FIG. 11).

The learned model 281 is previously produced by a training apparatus provided separately from the image processing apparatus 200b, and stored in the storage 8 similar to the learned model 81 in the first embodiment. In other words, similar to the learned model 81 in the first embodiment, the learned model 281 is produced by machine learning using deep learning to learn image conversion (image processing) that suppresses an image of ribs in the bone-present tomographic image 21 as an input.

The image output 95 (image processor 209) is configured to direct the display 7 to display the bone-suppressed tomographic image 220 generated and the bone-present tomographic image 21 generated adjacent to each other similar to the first embodiment.

The adjustment processing unit 294 (image processor 209) is configured to perform adjustment of the suppression degree of the bone structure in the bone-suppressed tomographic image 220, which is generated by the bone suppression processing unit 292, on the bone-suppressed tomographic image 220 similar to the adjustment processing unit 94 in the first embodiment. In the second embodiment, the adjustment processing unit 294 is configured to adjust the suppression degree of the bone structure in the bone-suppressed tomographic image 220 based on the bone-present tomographic image 21, which is a pre-bone-suppression image before the bone structure is suppressed by the bone suppression processing unit 292, and the bone-suppressed tomographic image 220, which is a post-bone-suppression image after the bone structure is suppressed by the bone suppression processing unit 292.

In the second embodiment, the adjustment factor K is acquired in accordance with an input instruction accepted by the input acceptor 6 similar to the first embodiment. Subsequently, the adjustment processing unit 294 adjusts the suppression degree of the bone structure in the bone-suppressed tomographic image 220 in accordance with the adjustment factor K acquired. Firstly, the adjustment processing unit 294 generates a bone-extracted tomographic image 222 by acquiring differences between the bone-present tomographic image 21 generated and the bone-suppressed tomographic image 20 generated. The bone-extracted tomographic image 222 is an image in which only components of the bone structure are extracted from the bone-present tomographic image 21.

Subsequently, the adjustment processing unit 294 performs constant multiplication on the generated bone-extracted tomographic image 222 similar to the first embodiment. The adjustment processing unit 294 generates an adjusted bone-extracted tomographic image 222a by multiplying a pixel value of each pixel included in the generated bone-extracted tomographic image 222 by the adjustment factor K. Subsequently, the adjustment processing unit 294 acquires differences between the adjusted bone-extracted tomographic image 222a generated and the bone-present tomographic image 21 whereby generating the adjusted bone-suppressed tomographic image 220a, which is the bone-suppressed tomographic image 220 whose suppression degree of the bone structure is adjusted. The image output 95 is configured to direct the display 7 to display the adjusted bone-suppressed tomographic image 220a generated and the bone-present tomographic image 21 adjacent to each other similar to the first embodiment.

The other configuration of the second embodiment is similar to the configuration of the first embodiment.

### Advantages of Second Embodiment

In the second embodiment, the following advantages are obtained.

In the second embodiment, as discussed above, the bone suppression processing unit 292 (image processor 209) is configured to suppress the bone structure (ribs) in the tomographic image (bone-present tomographic image 21) after the reconstruction is performed. In the second embodiment, as discussed above, the reconstruction processing unit 293 (image processor 209) is configured to generate a bone-present tomographic image 21, which is a tomographic image including the bone structure of the thorax (target part), by performing the reconstruction on the plurality of X-ray images 10 captured; and the bone suppression processing unit 292 (image processor 209) is configured to generate the bone-suppressed tomographic image 220 by suppressing the ribs (bone structure) on the bone-present tomographic image 21, which is generated by the reconstruction processing unit 293; and the adjustment processing unit 294 (image processor 209) is configured to adjust the suppression degree of the bone structure in the bone-suppressed tomographic image 220, which is generated by the bone suppression processing unit 292. According to this configuration, appearance of the bone structure can be more accurately suppressed by adjusting a suppression degree of the bone structure in the generated bone-suppressed tomographic image 220 if the suppression degree of the bone structure is too large or too small similar to the first embodiment. Consequently, it is possible to further prevent deterioration of visibility of the target part in the generated bone-suppressed tomographic image 220 (adjusted bone-suppressed tomographic image 220a). The other advantages of the second embodiment are similar to the advantages of the first embodiment.

### Third Embodiment

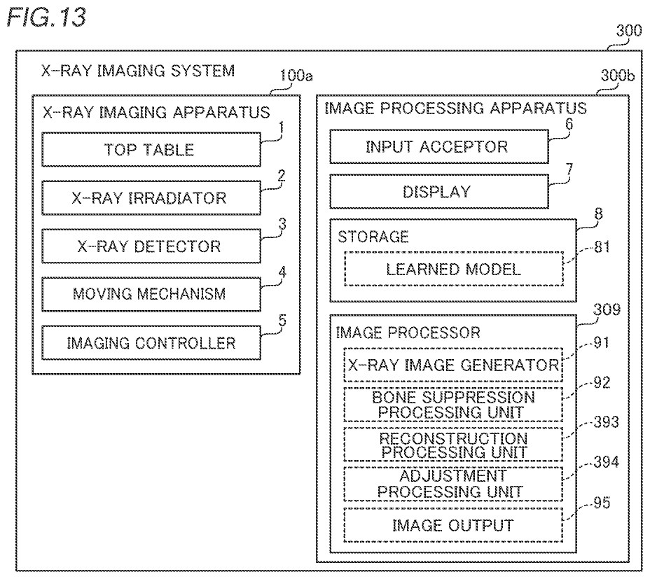

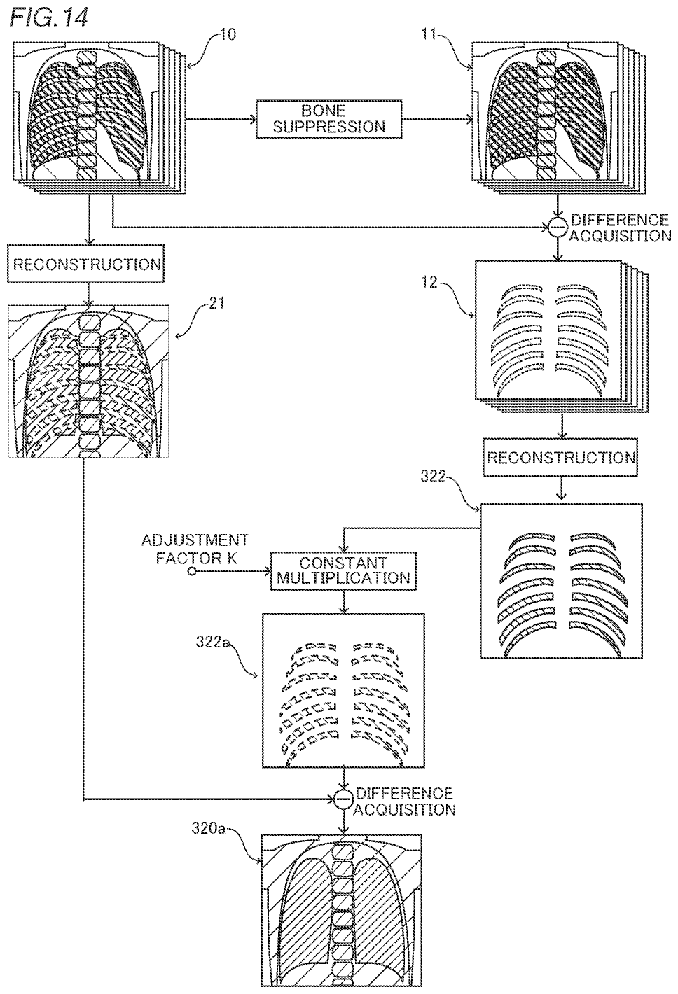

An X-ray imaging system 300 according to a third embodiment is now described with reference to FIGS. 13 and 14. In the third embodiment, the suppression degree of the bone structure is adjusted in the bone-suppressed tomographic image 20 based on a bone-present tomographic image 21, which in a tomographic image after reconstruction is performed, and a bone-extracted tomographic image 322, which is a tomographic image in which the bone structure is extracted. The same configurations in the third embodiment as those of the first embodiment are denoted by the same reference numerals, and their description is omitted.

As shown in FIG. 13, the X-ray imaging system 300 according to the third embodiment includes an X-ray imaging apparatus 100a, and an image processing apparatus 300b. The image processing apparatus 300b includes an image processor 309. Similar to the image processor 9 in the first embodiment, the image processor 309 is configured to generate a bone-suppressed tomographic image 20 based on X-ray images 10. Also, the image processor 309 includes an X-ray image generator 91, a bone suppression processing unit 92, a reconstruction processing unit 393, an adjustment processing unit 394, and an image output 95 as functional components.

As shown in FIG. 14, the X-ray image generator 91 (image processor 309) generates 41 X-ray images 10 based on the detection signals (image signals) of images captured (detected) by X-ray imaging (tomosynthesis imaging) by using the X-ray imaging apparatus 100a similar to the first embodiment. Similar to the first embodiment, the bone suppression processing unit 92 (image processor 309) is configured to generate a plurality of bone-suppressed X-ray images 11 by suppressing the bone structure (bone suppression) using a learned model 81 on a plurality of X-ray images 10 generated.

Similar to the first embodiment, the reconstruction processing unit 393 (image processor 309) is configured to perform reconstruction on the plurality of bone-suppressed X-ray images 11 whereby generating a bone-suppressed tomographic image 20 (see FIG. 3) similar to the first embodiment. Also, similar to the first embodiment, the reconstruction processing unit 393 is configured to perform reconstruction on the plurality of X-ray images 10 whereby generating a bone-present tomographic image 21. In the third embodiment, in order to adjust the suppression degree of the bone structure in the bone-suppressed tomographic image 20 to be generated, the reconstruction processing unit 393 is configured to perform the reconstruction on a plurality of bone-extracted images 12 (see FIG. 14) generated by an adjustment processing unit 394, which will be described later, whereby generating a bone-extracted tomographic image 322 (see FIG. 14). The bone-extracted tomographic image 322 is a tomographic image in which only components of the bone structure of the subject 101 are extracted. Processes of the reconstruction are similar to the first embodiment.

The image output 95 (image processor 309) is configured to direct the display 7 to display the bone-suppressed tomographic image 20 generated and the bone-present tomographic image 21 generated adjacent to each other similar to the first embodiment.

The adjustment processing unit 394 (image processor 309) is configured to perform adjustment of the suppression degree of the bone structure in the bone-suppressed tomographic image 20, is which generated by the reconstruction processing unit 393, on the bone structure in the bone-suppressed tomographic image 20 similar to the adjustment processing unit 94 in the first embodiment.

In the third embodiment, the adjustment factor K is acquired in accordance with an input instruction accepted by the input acceptor 6 similar to the first embodiment. Subsequently, the adjustment processing unit 394 adjusts the suppression degree of the bone structure in the bone-suppressed tomographic image 20 in accordance with the adjustment factor K acquired. Firstly, similar to the first embodiment, the adjustment processing unit 394 generates the plurality of bone-extracted images 12 based on the plurality of X-ray images 10, and the plurality of bone-suppressed X-ray images 11, which are the plurality of X-ray images 10 in which the bone structure is suppressed by the bone suppression processing unit 92. Specifically, the plurality of bone-extracted images 12 are generated by acquiring differences between the plurality of X-ray images 10 and the plurality of bone-suppressed X-ray images 11 by the adjustment processing unit 394. The bone-extracted images 12 are images of the extracted bone structure in the thorax of the subject 101.

Subsequently, the adjustment processing unit 394 performs constant multiplication on the bone-extracted tomographic image 322 generated by performing reconstruction on the bone-extracted image 12 by the reconstruction processing unit 393 similar to the first embodiment. Specifically, the adjustment processing unit 394 generates an adjusted bone-extracted tomographic image 322a by multiplying a pixel value of each pixel included in the generated bone-extracted tomographic image 322 by the adjustment factor K.

In the third embodiment, the adjustment processing unit 394 is configured to generate an adjusted bone-suppressed tomographic image 320a, which is the bone-suppressed tomographic image 20 in which the suppression degree of the bone structure is adjusted based on the bone-present tomographic image 21 and the bone-extracted tomographic image 322. Specifically, the adjustment processing unit 394 acquires differences between the adjusted bone-extracted tomographic image 322a generated and the bone-present tomographic image 21 whereby generating the adjusted bone-suppressed tomographic image 320a. The image output 95 is configured to direct the display 7 to display the adjusted bone-suppressed tomographic image 320a generated and the bone-present tomographic image 21 adjacent to each other similar to the first embodiment.

The other configuration of the third embodiment is similar to the configuration of the first embodiment.

### Advantages of Third Embodiment

In the third embodiment, the following advantages are obtained.

In the third embodiment, as discussed above, the bone suppression processing unit 92 (image processor 309) is configured to perform the bone structure on each of the plurality of X-ray images 10 generated; the reconstruction processing unit 393 (image processor 309) is configured to generate a bone-present tomographic image 21 including the bone structure of the thorax (target part) by performing the reconstruction on the plurality of X-ray images 10, and generate a bone-extracted tomographic image 322, which is a tomographic image in which the bone structure of the thorax is extracted, by performing the reconstruction on a plurality of bone-extracted images 12 in which the bone structure of the thorax is extracted and which are generated based on the plurality of X-ray images 10 and the plurality of bone-suppressed X-ray images 11, which are the plurality of X-ray images 10 in which the bone structure is suppressed by the bone suppression processing unit 92; and the adjustment processing unit 394 (image processor 309) is configured to generate an adjusted bone-suppressed tomographic image 320a in which the suppression degree of the bone structure is adjusted based on the bone-present tomographic image 21 and the bone-extracted tomographic image 322. According to this configuration, appearance of the bone structure can be more accurately suppressed by adjusting a suppression degree of the bone structure in the generated bone-suppressed tomographic image 20 if the suppression degree of the bone structure is too large or too small similar to the first embodiment. Consequently, it is possible to further prevent deterioration of visibility of the target part in the generated bone-suppressed tomographic image 20 (adjusted bone-suppressed tomographic image 320a). The other advantages of the third embodiment are similar to the advantages of the first embodiment.

### Modified Embodiments

Note that the embodiment disclosed this time must be considered as illustrative in all points and not restrictive. The scope of the present invention is not shown by the above description of the embodiments but by the scope of claims for patent, and all modifications (modified embodiments) within the meaning and scope equivalent to the scope of claims for patent are further included.

While the example in which the image processor 9 (209, 309) is configured to generate the bone-suppressed tomographic image 20 (220) in which the bone structure including ribs in the thorax (chest and abdomen) is suppressed has been shown in the aforementioned first to third embodiments, the present invention is not limited to this. In the present invention, the image processor 9 (209, 309) may be configured to generate the bone-suppressed tomographic image in which the bone structure including ribs is suppressed in the chest or abdomen. Also, in the present invention, the image processor 9 (209, 309) may be configured to generate the bone-suppressed tomographic image in which the femur is suppressed in angiography of a leg. Also, the image processor 9 (209, 309) may be configured to suppress a spine and clavicles in addition to the ribs in the thorax.

While the example in which the bone-suppressed tomographic image 20 (220) in which the bone structure (ribs) is suppressed is generated by performing image processing using the learned model 81 (281) produced by machine learning has been shown in the aforementioned first to third embodiments, the present invention is not limited to this. For example, the bone part may be extracted by an algorithm that is produced based on a rule (e.g., template matching) to suppress the bone structure. Also, DES (dual energy subtraction) may be used to suppress the bone structure based on X-ray images generated by using X-rays having two different energies.

While the example in which the bone structure (ribs) is suppressed by using the common learned model 81 in each of the 41 X-ray images 10 has been shown in the aforementioned first and third embodiments, the present invention is not limited to this. For example, the bone structure may be suppressed by using a plurality of learned models that have learned to suppress the bone structure corresponding to different imaging positions (irradiation angles). In this configuration, 41 different learned models corresponding to different irradiation angles can be used to suppress the bone structure in the 41 X-ray images 10. Alternatively, four or five learned models each of which corresponds to a plurality of irradiation angles can be switched from one learned model to another.

While the example in which the image processing apparatus 100b (200b, 300b) includes the display 7 configured to display the bone-suppressed tomographic image 20 (220) and the adjusted bone-suppressed tomographic image 20a (220a, 320a) has been shown in the aforementioned first to third embodiments, the present invention is not limited to this. For example, the bone-suppressed tomographic image 20 (220) generated and the adjusted bone-suppressed tomographic image 20a (220a, 320a) generated may be displayed on a display included in the X-ray imaging apparatus 100a. Also, the bone-suppressed tomographic image 20 (220) generated and the adjusted bone-suppressed tomographic image 20a (220a, 320a) generated may be output to an external display separated from the image processing apparatus 100b (200b, 300b) to display the bone-suppressed tomographic image 20 (220) and the adjusted bone-suppressed tomographic image 20a (220a, 320a).

While the example in which the bone-present tomographic image 21, and the bone-suppressed tomographic image 20 (220) or the adjusted bone-suppressed tomographic image 20a (220a, 320a) are displayed adjacent to each other on the display 7 has been shown in the aforementioned first to third embodiments, the present invention is not limited to this. For example, the X-ray image 10 may be displayed adjacent to the bone-suppressed tomographic image 20 (220) or the adjusted bone-suppressed tomographic image 20a (220a, 320a). Also, the X-ray images 10 in which the bone structure is not suppressed, and the bone-suppressed X-ray image 11 in which the bone structure is suppressed may be displayed adjacent to each other on the display 7. Alternatively, the display may switch between the bone-suppressed tomographic image 20 (220) and the adjusted bone-suppressed tomographic image 20a (220a, 320a) in accordance with an input instruction accepted by the input acceptor 6 to display one of them.

While the example in which the imaging controller 5 and the image processor 9 (209, 309), which are constructed as separated hardware components, are configured to perform control processing for X-ray imaging and control processing for generating the bone-suppressed tomographic image 20 (220) and the adjusted bone-suppressed tomographic image 20a (220a, 320a), respectively, has been shown in the aforementioned first to third embodiments, the present invention is not limited to this. For example, one common controller (hardware) may be configured to perform the control processing for X-ray imaging and the processing for generating the bone-suppressed tomographic image 20 (220) and the adjusted bone-suppressed tomographic image 20a (220a, 320a). In this configuration, X-ray imaging (tomosynthesis imaging) and image processing for generating the bone-suppressed tomographic image 20 (220) and the adjusted bone-suppressed tomographic image 20a (220a, 320a) may be performed by a single apparatus.

While the example in which the X-ray image generator 91, the bone suppression processing unit 92 (292), the reconstruction processing unit 93 (293, 393), the adjustment processing unit 94 (294, 394) and the image output 95 are configured to serve as functional blocks (software) in a single hardware component (image processor 9) has been shown in the aforementioned first to third embodiments, the present invention is not limited to this. For example, the X-ray image generator 91, the bone suppression processing unit 92 (292), the reconstruction processing unit 93 (293, 393), the adjustment processing unit 94 (294, 394) and the image output 95 may be constructed of separated hardware components (calculation circuits).

While the example in which tomosynthesis imaging is performed on the thorax (target part) of the subject 101 while moving the X-ray irradiator 2 and the X-ray detector 3 has been shown in the aforementioned first to third embodiments, the present invention is not limited to this. For example, tomosynthesis imaging is performed while moving only the X-ray irradiator 2.

While the example in which the X-ray irradiator 2 is held by the irradiator holder 4a having a columnar shape has been shown in the aforementioned first to third embodiments, the present invention is not limited to this. For example, the X-ray irradiator 2 may be a movable ceiling-mounted type irradiator. Alternatively, the X-ray irradiator 2 and the X-ray detector 3 may be held by a C-arm.

While the example in which the X-ray irradiator 2 is configured to move from a position of −20 degrees to a position of +20 degrees with respect to the subject 101 as the vertical direction defined as a reference (zero degree) in tomosynthesis imaging has been shown in the aforementioned first to third embodiments, the present invention is not limited to this. For example, the X-ray irradiator 2 may be configured to move from a position of −15 degree to a position of +15 degrees.

While the example in which an X-ray image of the subject is captured at every one-degree movement of the X-ray irradiator 2 whereby capturing 41 X-ray images 10 of the subject in the tomosynthesis imaging has been shown in the aforementioned first to third embodiments, the present invention is not limited to this. For example, an X-ray image of the subject may be captured at every two-degree movement or every 0.5-degree movement of the X-ray irradiator 2.

While the example in which the input acceptor 6 configured to accept an input instruction to set an adjustment factor K for adjusting the suppression degree of the bone structure is provided has been shown in the aforementioned first to third embodiments, the present invention is not limited to this. For example, a predetermined adjustment factor K may be used to adjust the suppression degree of the bone structure.

While the example in which the bone suppression or the reconstruction is performed on a plurality of X-ray images 10 acquired by tomosynthesis imaging has been shown in the aforementioned first to third embodiments, the present invention is not limited to this. For example, preprocessing for adjusting image quality before the bone suppression is performed on the plurality of X-ray images 10 captured by the tomosynthesis imaging. Specifically, contrast adjustment, resolution adjustment, noise removal, etc. may be performed as the preprocessing on the X-ray images 10 captured. In a case in which preprocessing is performed on the X-ray images 10, parameters of the preprocessing may be configured to be adjusted by users.

While the example in which an area corresponding to a lung field in the bone-suppressed tomographic image 20 (220) is specified as the adjustment area 20r for adjusting the suppression degree of the bone structure in each pixel based on an input instruction by accepted by the input acceptor 6 has been shown in the aforementioned first to third embodiments, the present invention is not limited to this. For example, not the entire lung field but only a part of the lung field (e.g., only right lung) may be specified as the adjustment area 20r. Alternatively, the suppression degree can be adjusted in each pixel so that the adjustment factor K is reduced with a distance from coordinates that are specified based on the input instruction accepted by the input acceptor 6 whereby concentrically spreading different suppression degrees.

While the example in which input instructions can be accepted to specify the adjustment area 20r and the adjustment factor K when the bone-suppressed tomographic image 20 (220) is displayed on the display 7 has been shown in the aforementioned first to third embodiments, the present invention is not limited to this. For example, input instructions from an operator such as a doctor can be accepted to specify the adjustment area 20r and the adjustment factor K when a tomographic image in which the bone structure is not suppressed (bone-present tomographic image 21) is displayed on the display 7.

### Modes

The aforementioned exemplary embodiment will be understood as concrete examples of the following modes by those skilled in the art.

### Mode Item 1

An X-ray imaging system includes an X-ray irradiator configured to irradiate a target part of a subject with X-rays; an X-ray detector configured to detect the X-rays with which the subject is irradiated by the X-ray irradiator; a moving mechanism configured to move at least one of the X-ray irradiator and the X-ray detector; an imaging controller configured to perform X-ray imaging of the target part of the subject while the moving mechanism is moving the at least one of the X-ray irradiator and the X-ray detector; and an image processor configured to generate, based on a plurality of X-ray images generated by the X-ray imaging, a bone-suppressed tomographic image representing a cross-section of the subject in which a bone structure of the target part is suppressed, wherein the image processor includes a bone suppression processing unit configured to suppress the bone structure of the target part based on the plurality of X-ray images generated, a reconstruction processing unit configured to perform, based on the plurality of X-ray images generated, reconstruction for generating the tomographic image, and an adjustment processing unit configured to adjust a suppression degree of the bone structure in the bone-suppressed tomographic image to be generated.

### Mode Item 2

In the X-ray imaging system according to mode item 1, the bone suppression processing unit is configured to suppress the bone structure on the plurality of X-ray images before the reconstruction is performed or on the tomographic image after the reconstruction is performed; and the adjustment processing unit is configured to adjust the suppression degree of the bone structure in the bone-suppressed tomographic image to be generated by performing adjustment of the suppression degree of the bone structure based on a post-bone-suppression image that is a result of the suppression by the bone suppression processing unit.

### Mode Item 3

In the X-ray imaging system according to mode item 2, the adjustment processing unit is configured to adjust the suppression degree of the bone structure in the bone-suppressed tomographic image based on a pre-bone-suppression image before the bone structure is suppressed by the bone suppression processing unit and the post-bone-suppression image after the bone structure is suppressed by the bone suppression processing unit.

### Mode Item 4

In the X-ray imaging system according to any of mode items 1 to 3, an input acceptor configured to accept an input instruction to adjust the suppression degree of the bone structure is further provided; and the adjustment processing unit is configured to adjust the suppression degree of the bone structure in the bone-suppressed tomographic image based on a predetermined adjustment factor and to set the adjustment factor based on the input instruction accepted by the input acceptor.

### Mode Item 5

In the X-ray imaging system according to any of mode items 1 to 4, the adjustment processing unit is configured be able to adjust the suppression degree of the bone structure in each of pixels of the bone-suppressed tomographic image to be generated.

### Mode Item 6

In the X-ray imaging system according to any of mode items 1 to 5, the bone suppression processing unit is configured to suppress the bone structure on each of the plurality of X-ray images generated; the adjustment processing unit is configured to perform the adjustment of the suppression degree of the bone structure on the plurality of X-ray images in which the bone structure is suppressed by the bone suppression processing unit; and the reconstruction processing unit configured to generate the bone-suppressed tomographic image in which the suppression degree of the bone structure is adjusted by performing the reconstruction based on the plurality of X-ray images in which the suppression degree of the bone structure is adjusted by the adjustment processing unit.

### Mode Item 7

In the X-ray imaging system according to any of mode items 1 to 5, the reconstruction processing unit is configured to generate a bone-present tomographic image that is the tomographic image including the bone structure of the target part by performing the reconstruction on the plurality of X-ray images; the bone suppression processing unit is configured to generate the bone-suppressed tomographic image by suppressing the bone structure on the bone-present tomographic image, which is generated by the reconstruction processing unit; and the adjustment processing unit is configured to perform the adjustment of the suppression degree of the bone structure on the bone-suppressed tomographic image, which is generated by the bone suppression processing unit.

### Mode Item 8

In the X-ray imaging system according to any of mode items 1 to 5, wherein the bone suppression processing unit is configured to suppress the bone structure on each of the plurality of X-ray images generated; the reconstruction processing unit is configured to generate a bone-present tomographic image that is the tomographic image including the bone structure of the target part by performing the reconstruction on the plurality of X-ray images, and to generate a bone-extracted tomographic image that is the tomographic image in which the bone structure of the target part is extracted by performing the reconstruction on a plurality of bone-extracted images in which the bone structure of the target part is extracted and which are generated based on the plurality of X-ray images and the plurality of X-ray images in which the bone structure is suppressed by the bone suppression processing unit; and the adjustment processing unit is configured to generate the bone-suppressed tomographic image in which the suppression degree of the bone structure is adjusted based on the bone-present tomographic image and the bone-extracted tomographic image.

### Mode Item 9

In the X-ray imaging system according to any of mode items 1 to 8, the reconstruction processing unit is configured to generate a bone-present tomographic image that is the tomographic image including the bone structure of the target part by performing the reconstruction on the plurality of X-ray images; and a display configured to display the bone-present tomographic image including the bone structure, and the bone-suppressed tomographic image in which the bone structure is suppressed is further provided.

### Mode Item 10

In the X-ray imaging system according to any of mode items 1 to 9, the target part includes at least one of a chest and an abdomen of the subject; and the image processor is configured to generate the bone-suppressed tomographic image in which the bone structure including ribs in the at least one of the chest and the abdomen is suppressed.

### Mode Item 11

In the X-ray imaging system according to any of mode items 1 to 10, the bone suppression processing unit is configured to suppress the bone structure of the target part by performing image processing by using a learned model that is produced by machine learning to suppress the bone structure.

### Mode Item 12

An image processing method includes a step of moving at least one of an X-ray irradiator configured to irradiate a target part of a subject with X-rays and an X-ray detector configured to detect the X-rays with which the subject is irradiated by the X-ray irradiator, and generating a plurality of X-ray images by performing X-ray imaging on the target part of the subject during movement of the at least one of the X-ray irradiator and the X-ray detector; and a step of generating a bone-suppressed tomographic image representing a cross-section of the subject in which a bone structure of the target part is suppressed based on the plurality of X-ray images generated by performing the X-ray imaging, wherein the step of generating the bone-suppressed tomographic image includes a step of suppressing the bone structure of the target part based on the plurality of X-ray images generated, a step of performing, based on the plurality of X-ray images generated, reconstruction for generating the tomographic image, and a step of adjusting a suppression degree of the bone structure in the bone-suppressed tomographic image to be generated.

### DESCRIPTION OF REFERENCE NUMERALS

2; X-ray irradiator
3; X-ray detector
4; moving mechanism
5; imaging controller
6; input acceptor
7; display
9, 209, 309; image processor
10; X-ray image (pre-bone-suppression image)
11; bone-suppressed X-ray image (post-bone-suppression image)
12; bone-extracted image
20; bone-suppressed tomographic image
20a, 220a, 320a; adjusted bone-suppressed tomographic image
21; bone-present tomographic image (pre-bone-suppression image)
81, 281; learned model
92, 292; bone suppression processing unit
93, 293, 393; reconstruction processing unit
94, 294, 394; adjustment processing unit
100, 200, 300; X-ray imaging system
101; subject
220; bone-suppressed tomographic image (post-bone-suppression image)
322; bone-extracted tomographic image

1. An X-ray imaging system comprising:
an X-ray irradiator configured to irradiate a target part of a subject with X-rays;
an X-ray detector configured to detect the X-rays with which the subject is irradiated by the X-ray irradiator;
a moving mechanism configured to move at least one of the X-ray irradiator and the X-ray detector;
an imaging controller configured to perform tomosynthesis imaging of the target part of the subject while the moving mechanism is moving the at least one of the X-ray irradiator and the X-ray detector; and
an image processor configured to generate, based on a plurality of X-ray images generated by the tomosynthesis imaging, a bone-suppressed tomographic image representing a cross-section of the subject in which a bone structure of the target part is suppressed, wherein
the image processor includes
a bone suppression processing unit configured to suppress the bone structure of the target part based on the plurality of X-ray images generated,
a reconstruction processing unit configured to perform, based on the plurality of X-ray images generated, reconstruction for generating the tomographic image, and
an adjustment processing unit configured to adjust a suppression degree of the bone structure in the bone-suppressed tomographic image to be generated.

2. The X-ray imaging system according to claim 1, wherein
the bone suppression processing unit is configured to suppress the bone structure on the plurality of X-ray images before the reconstruction is performed or on the tomographic image after the reconstruction is performed; and
the adjustment processing unit is configured to adjust the suppression degree of the bone structure in the bone-suppressed tomographic image to be generated by performing adjustment of the suppression degree of the bone structure based on a post-bone-suppression image that is a result of the suppression by the bone suppression processing unit.

3. The X-ray imaging system according to claim 2, wherein the adjustment processing unit is configured to adjust the suppression degree of the bone structure in the bone-suppressed tomographic image based on a pre-bone-suppression image before the bone structure is suppressed by the bone suppression processing unit and the post-bone-suppression image after the bone structure is suppressed by the bone suppression processing unit.

4. The X-ray imaging system according to claim 1 further comprising an input acceptor configured to accept an input instruction to adjust the suppression degree of the bone structure, wherein
the adjustment processing unit is configured to adjust the suppression degree of the bone structure in the bone-suppressed tomographic image based on a predetermined adjustment factor and to set the adjustment factor based on the input instruction accepted by the input acceptor.

5. The X-ray imaging system according to claim 1, wherein the adjustment processing unit is configured be able to adjust the suppression degree of the bone structure in each of pixels of the bone-suppressed tomographic image to be generated.

6. The X-ray imaging system according to claim 1, wherein
the bone suppression processing unit is configured to suppress the bone structure on each of the plurality of X-ray images generated;
the adjustment processing unit is configured to perform the adjustment of the suppression degree of the bone structure on the plurality of X-ray images in which the bone structure is suppressed by the bone suppression processing unit; and
the reconstruction processing unit configured to generate the bone-suppressed tomographic image in which the suppression degree of the bone structure is adjusted by performing the reconstruction based on the plurality of X-ray images in which the suppression degree of the bone structure is adjusted by the adjustment processing unit.

7. The X-ray imaging system according to claim 1, wherein
the reconstruction processing unit is configured to generate a bone-present tomographic image that is the tomographic image including the bone structure of the target part by performing the reconstruction on the plurality of X-ray images;
the bone suppression processing unit is configured to generate the bone-suppressed tomographic image by suppressing the bone structure on the bone-present tomographic image, which is generated by the reconstruction processing unit; and
the adjustment processing unit is configured to perform the adjustment of the suppression degree of the bone structure on the bone-suppressed tomographic image, which is generated by the bone suppression processing unit.

8. The X-ray imaging system according to claim 1, wherein
the bone suppression processing unit is configured to suppress the bone structure on each of the plurality of X-ray images generated;
the reconstruction processing unit is configured to generate a bone-present tomographic image that is the tomographic image including the bone structure of the target part by performing the reconstruction on the plurality of X-ray images, and to generate a bone-extracted tomographic image that is the tomographic image in which the bone structure of the target part is extracted by performing the reconstruction on a plurality of bone-extracted images in which the bone structure of the target part is extracted and which are generated based on the plurality of X-ray images and the plurality of X-ray images in which the bone structure is suppressed by the bone suppression processing unit; and
the adjustment processing unit is configured to generate the bone-suppressed tomographic image in which the suppression degree of the bone structure is adjusted based on the bone-present tomographic image and the bone-extracted tomographic image.

9. The X-ray imaging system according to claim 1, wherein
the reconstruction processing unit is configured to generate a bone-present tomographic image that is the tomographic image including the bone structure of the target part by performing the reconstruction on the plurality of X-ray images; and
the X-ray imaging system further comprises a display configured to display the bone-present tomographic image including the bone structure, and the bone-suppressed tomographic image in which the bone structure is suppressed.

10. The X-ray imaging system according to claim 1, wherein
the target part includes at least one of a chest and an abdomen of the subject; and
the image processor is configured to generate the bone-suppressed tomographic image in which the bone structure including ribs in the at least one of the chest and the abdomen is suppressed.

11. The X-ray imaging system according to claim 1, wherein the bone suppression processing unit is configured to suppress the bone structure of the target part by performing image processing by using a learned model that is produced by machine learning to suppress the bone structure.

12. An image processing method comprising:
a step of moving at least one of an X-ray irradiator configured to irradiate a target part of a subject with X-rays and an X-ray detector configured to detect the X-rays with which the subject is irradiated by the X-ray irradiator, and generating a plurality of X-ray images by performing tomosynthesis imaging on the target part of the subject during movement of the at least one of the X-ray irradiator and the X-ray detector; and
a step of generating a bone-suppressed tomographic image representing a cross-section of the subject in which a bone structure of the target part is suppressed based on the plurality of X-ray images generated by performing the tomosynthesis imaging, wherein
the step of generating the bone-suppressed tomographic image includes
a step of suppressing the bone structure of the target part based on the plurality of X-ray images generated,
a step of performing, based on the plurality of X-ray images generated, reconstruction for generating the tomographic image, and
a step of adjusting a suppression degree of the bone structure in the bone-suppressed tomographic image to be generated.
